(19) United States
(12) Patent Application Publication
(10) Pub. No.: US 2018/0036167 A1
(43) Pub. Date: Feb. 8, 2018

(54) IRIS COLOR CHANGING METHOD

(71) Applicant: Faith KENAR, Umraniye/Istanbul (TR)

(72) Inventor: Fatih KENAR, Umraniye/Istanbul (TR)

(21) Appl. No.: 15/549,993

(22) PCT Filed: Aug. 16, 2016
(86) PCT No.: PCT/TR2016/000119

§ 371 Date: Aug. 9, 2017

(30) Foreign Application Priority Data
Aug. 28, 2015 (TR) 2015/10707

(51) Int. Cl. A61F 9/008

(52) U.S. Cl. CPC A61F 9/008; A61F 2009/00876

(57) ABSTRACT

The invention is related to iris color changing method that ensures a color change in iris by shedding the layer that contain the cells with melanin pigments in the upper superficial part and stroma of the iris, colored part of the eye, through photo-disruptive effect.

### TECHNICAL FIELD

Invention is related to an iris color changing method that changes the color of iris by diffusing the layer which has the cells that contains melanin pigments on upper surface of the iris, colored part of the eye, and its stroma by means of photodisruptive effect.

### KNOWN STATUS OF THE TECHNIQUE

Throughout history, people, especially women, care about their appearance. Thus, everything that affects appearance has been subjected to development. For example, clothes are subjected to a regular process named fashion. People change their appearance, especially the visible parts of their bodies such as noses with the help of surgical operations. Aim of these operations is to make women seem more beautiful and men handsomer. Such demands from people make innovations in medical science necessary.

Everybody knows that eye color is a parameter that indicates the beauty of a woman and good looks of a man. Thus, contact lens technology was developed. Aim of the contact lens technology is to change the eye color with the help of a foreign object put on the eye. A person is able to achieve their desired eye color by putting on contact lenses. There are several problems in using contact lenses. Those problems are: difficulty in putting on and removing contact lenses and it is time consuming; possibility of infection in the eye during these processes; possibility of dry eye syndrome which causes eye burning, stinging pain and redness in long term uses. Besides contact lenses may cause a dull appearance that does not seems natural with the effect of the original eye color.

In order to eliminate the above indicated problems of lens technology, nowadays laser devices shed the layer on anterior superficial and deep part of the iris that include the melanin containing pigments through photodisruptive effect and stroma is uncovered. Stroma consists of veins, muscle and collagen tissue. Eye color is gray due to the muscle and collagen tissue. After the laser operation, eye color can be changed into light brown, hazel, green, blue or green according to the melanin density in stroma and in pigment epithelium layer behind the iris.

Sphincter and dilatators muscles of the iris can be affected while implementing the laser surgery or after that and therefore pupil function of the eye may be deteriorated. As a result of this, temporary enlargement of the pupil may be experienced. Enlargement of the pupil may cause sensibility to sun and also farsightedness problems in people who are forty or more years old.

When laser operations are implemented with 315 nanometers and lower wave lengths, they are absorbed in cornea (i.e. 315 nanometers and lower wave lengths cannot get through the cornea) thus there is no color change in iris. If the laser with 315 nanometers is implemented to iris within the eye by getting through the cornea, that may result in cataract development.

Today, when an operation is implemented by means of a laser device with 420 nanometers wave length, that may result in bleeding because melanin absorption coefficients and hemoglobin absorption coefficient are close to each other. When bleeding occurs, that area is not subjected to the laser beam in order to prevent a second bleeding, thus pigments cannot be diffused for that session and operation period is extended.

Today, when an operation is implemented by means of a laser device with 540 nanometers wave length, that may result in bleeding because melanin absorption coefficients and hemoglobin absorption coefficient are close to each other. When bleeding occurs, that area is not subjected to the laser beam in order to prevent a second bleeding, thus pigments cannot be diffused for that session and operation period is extended.

Today, when an operation is implemented by means of a laser device with 580 nanometers wave length, that may result in bleeding because melanin absorption coefficients and hemoglobin absorption coefficient are close to each other. When bleeding occurs, that area is not subjected to the laser beam in order to prevent a second bleeding, thus pigments cannot be diffused for that session and operation period is extended.

Today, when an operation is implemented by means of a laser device with between 700 and 900 nanometers wave lengths, retina can be reached by getting through all tissues of the eye, because penetration hole, transmission is high in connection with optical window. Unpredictable retina damage may develop.

Today, when a laser beam is implemented with 1064 nanometers wave length and with penetration depth more than 300 micrometers, there may be bleeding; and if the pigment epithelium behind the iris is fractured it causes transillumination defect. This situation results in sensitivity to light and sun.

### DESCRIPTION OF THE INVENTION

The invention eliminates all the disadvantages that were stated above.

Method that is developed with the invention eliminates the usage difficulties of contact lenses, infection problem on the eye and dry eye syndrome due to long-term use of contact lens.

Besides, new color of the eye has the natural looks of the old color in illuminated, lightened spaces thanks to the method developed with the invention. Dull looking caused by lens usage is eliminated.

In the method that is developed with the invention, damage to the crystalline lens in pupil area is prevented by avoiding laser beams in 2 mm of pupil area which is shrunk by dripping pilosed.

In the invention it's avoided to apply 1000 and/or 2000 micrometer spot diameter laser beam to Pupillary iris (pupillary area of the iris) and Ciliary iris (ciliary area of the iris) with laser device at 532 nanometer wavelength, to apply 1064 nanometer wavelength, spot diameter 8 microns with a penetration depth above 300 microns to disrupt the superficial layer of Pupillary iris and Ciliary iris, dysfunction of pupil through protection of iris sphincter and dilator muscle tissue and correspondingly sun sensitivity based on enlargement of pupilla after laser beam application and development of near vision problems for people over the age of forty and cataract formation through use of crystalline lens. Bleeding risk is reduced by protecting blood veins with a careful laser operation on papilla edges, collarette area (Major arterial circle) and iris area at 3 and 9 o'clock. In case of bleeding during laser operation, bleeding can be stopped with a short-term pressure by the object lens that is used during laser. Because the operations are not implemented by a laser device with 315 nanometers and lower wave lengths in the method that is developed with the invention, there is no cornea absorption. Because the operations are not implemented by a laser with 315 nanometers and lower wave lengths, to the iris within the eye by getting through the cornea, probability of cataract is eliminated.

Because the operations are not implemented on 420 nanometers wave lengths that hemoglobin and melanin pigment absorption is similar (close) to each other, possible bleeding risk due to the usage of this wave length is eliminated in the method that is developed with the invention.

Because the operations are not implemented on 540 nanometers wave lengths that hemoglobin and melanin pigment absorption is similar (close) to each other, possible bleeding risk due to the usage of this wave length is eliminated in the method that is developed with the invention.

Because the operations are not implemented on 580 nanometers wave lengths that hemoglobin and melanin pigment absorption is similar (close) to each other, possible bleeding risk due to the usage of this wave length is eliminated in the method that is developed with the invention.

Because the operations are not implemented on wave lengths between 700 and 900 nanometers, unpredictable retina damage that is caused by reaching the retina by getting through all tissues of the eye, because penetration hole and transmission is high in connection with optical window, is eliminated in the method that is developed with the invention.

Because the operations are implemented by a laser device with 1064 wave length and up to approximately 300 micrometers depth, problem of the pigment epithelium fracture behind the iris by the laser light and transillumination defect caused by this situation is eliminated in the method that is developed with the invention. Sun and light sensitivity due to transillumination defect on the eye that is subjected to the method of this invention is prevented. Bleeding risk is decreased.

A session for each eye lasts for approximately 5-10 minutes in the method of this invention. Thus a healthy color change in a short time is achieved with the method of this invention.

### EXPLANATION OF THE INVENTION

Iris color changing method developed with this invention was not developed for diagnosis or treatment purposes. Iris color changing method developed in this invention is for cosmetic and aesthetic purposes only. That is to say, a person with brown eyes or other colored eyes is not the cause of any disease. This method is for changing the color of eyes for aesthetic purposes only. The invention is a method for changing the color of the iris, prior to laser operation, pre-laser preparation (preoperative), laser application and post-laser (postoperative) to Irrigation Aspiration (I/A) operation of the eye under sterile conditions when required;
a—Twice 2% Pilokarpin HCl (Pilosed) with 5 minute intervals 15 minutes prior to laser application to create myosis effect and three times Proparakain HCl 0.5% (Alcaine) with 5 minute intervals 10 minutes prior for anesthesia are dropped. This way myosis and anesthesia is ensured. (Because iris evens out when Pilokarpin is dropped, it should be noted that a small decrease would occur in normal thickness).
b—When laser is applied to pupillary iris, collaret and ciliary iris, Yag laser capsulotomy lens shall be put on and when you want to apply laser to peripheral ciliary iris area the 1-2 mm part of the peripheral of iris starting from limbus or by observing the iris in more detail, it should be applied by putting on Yag laser iridotomy lens,
c—Avoiding laser beam in the pupil area,
d—Applying 532 nanometer wavelength 1000 micrometer spot diameter laser application to Pupillary iris (Pupillary area of iris),
e—Applying laser beam with 532 nanometer wavelength and 1000 micrometer spot diameter to Collarette (Swollen circle between pupillary and ciliary areas of the iris. Because in this area blood vessels are concentrated and anastomosed, in order to decrease bleeding) area,
f—If adequate pigment shedding and pupillary iris color lightening can't be achieved with 532 nanometer wavelength in Pupillary Iris and Collarette area; approach these areas carefully, don't approach the pupilla, focus on the anterior facet of iris, don't focus on the posterior facet pigment epithelium, penetration depth in which the laser beam effects shouldn't exceed 200-300 micrometers, leaving iris stromal cavity on the iris posterior facet that the laser doesn't effect to avoid break down of pupillary function at 200 micrometers, very carefully apply to the collarette area where blood vessel anastomosis are placed, laser beam shall be vertical to the iris and applied at 1064 nanometer wavelength at 8 micrometer spot diameter by decreasing the power of the laser and whilst directing the eye upwards, right, downwards and left (according to whichever clock dial it shall be done, the opposite clock dial shall be looked at),
g—Ciliary iris (Ciliary area of the iris) laser beam shall be vertical to the iris and applied at 1064 nanometer wavelength at 1064 wavelength, 8 micrometer spot diameter and penetration depth below 300 micrometers, and whilst directing the eye upwards, right, downwards and left (according to whichever clock dial it shall be done, the opposite clock dial shall be looked at) to break the superficial layer faster
h—When the number of pigments shedding from the superficial layer of ciliary iris to the front chamber is less than 25 large particles or 100 small cells in an area of 1 mm³, laser beam shall be applied to the deep layers of Ciliary Iris area at 532 nanometer wavelength, 2000 micrometer spot diameter in the same session,
i—When the number of pigments shedding from the superficial layer of ciliary iris to the front chamber is more than 25 large particles or 100 small cells, don't apply 2000 micrometer spot diameter laser beam at 532 nanometer wavelength to the deep layers of Ciliary Iris area in the same session, pause the laser operation and irrigate the iris pigments in the front chamber through Irrigation Aspiration (I/A) under sterile conditions with BSS Plus or Isolyte solution (BSS Plus contains: 4.6 mg Glutation disulphide Solution, 2.19 mg Sodium bicarbonate Solution, 0.433 mg Sodium phosphate Solution, 7.44 mg Sodium chloride Solution, 0.395 mg Potassium chloride Solution, 3.85 mg Calcium chloride dihydrate Solution, 23.0 mg Dextrose anhidrosis Solution, 5.0 mg Magnesium chloride hexahydrate Solution or Isolyte contains: Sodium acetate 3H₂O 0.64%, Sodium chloride 0.5%, Sodium citrate 0.075%, Potassium chloride 0.075%, Calcium chloride dihydrate 0.035%, Magnesium chloride 6H₂O 0.031%) (In this way glaucoma risk is decreased and it's ensured for the laser to achieve the tissue with more effectiveness in the next session).
j—Application of respectively 1000 and 2000 micrometers spot diameter laser beam with 532 nanometer wavelength in Pupillary and Ciliary area after I/A, consists of the steps above and takes approximately five to ten minutes per eye.

After steps (a, b, c, d, e, f, g, h, i and j) above, in a biomicroscopic examination on the eye applied with steps (a, b, c, d, e, f, g, h, i and j), if there are pigmented areas in the superficial layer and/or stroma of iris, steps required out of a, b, c, d, e, f, g, h, i and j are re-applied as necessary, the session is repeated until the pigmented areas disappear.

1. The invention is a method for iris color changing, it features; Pre-op preparation prior to laser application, laser application and when required irrigation of pigments in the front chamber of the eye after laser application through Irrigation Aspiration (I/A);
a—Twice 2% Pilokarpin HCl (Pilosed) with 5 minute intervals 15 minutes prior to laser application to create myosis effect and three times Proparakain HCl 0.5% (Alcaine) with 5 minute intervals 10 minutes prior for anesthesia are dropped. This way myosis and anesthesia is ensured. (Because iris evens out when Pilokarpin is dropped, it should be noted that a small decrease would occur in normal thickness.)
b—When laser is applied to pupillary iris, collaret and ciliary iris, Yag laser capsulotomy lens shall be put on and when you want to apply laser to peripheral ciliary iris area the 1-2 mm part of the peripheral of iris starting from limbus or by observing the iris in more detail, it should be applied by putting on Yag laser iridotomy lens,
c—Avoiding laser beam in the pupil area,
d—Applying 532 nanometer wavelength 1000 micrometer spot diameter laser application to Pupillary iris (Pupillary area of iris),
e—Applying laser beam with 532 nanometer wavelength and 1000 micrometer spot diameter to Collarette (Swollen circle between pupillary and ciliary areas of the iris. Because in this area blood vessels are concentrated and anastomosed, in order to decrease bleeding) area,
f—If adequate pigment shedding and pupillary iris color lightening can't be achieved with 532 nanometer wavelength in Pupillary Iris and Collarette area; approach these areas carefully, don't approach the pupilla, focus on the anterior facet of iris, don't focus on the posterior facet pigment epithelium, penetration depth in which the laser beam effects shouldn't exceed 200-300 micrometers, leaving iris stromal cavity on the iris posterior facet that the laser doesn't effect to avoid break down of pupillary function at 200 micrometers, very carefully apply to the collarette area where blood vessel anastomosis are placed, laser beam shall be vertical to the iris and applied at 1064 nanometer wavelength at 8 micrometer spot diameter by decreasing the power of the laser and whilst directing the eye upwards, right, downwards and left (according to whichever clock dial it shall be done, the opposite clock dial shall be looked at),
g—Ciliary iris (Ciliary area of the iris) laser beam shall be vertical to the iris and applied at 1064 nanometer wavelength at 1064 wavelength, 8 micrometer spot diameter and penetration depth below 300 micrometers, and whilst directing the eye upwards, right, downwards and left (according to whichever clock dial it shall be done, the opposite clock dial shall be looked at) to break the superficial layer faster,
h—When the number of pigments shedding from the superficial layer of ciliary iris to the front chamber is less than 25 large particles or 100 small cells in an area of 1 mm³, laser beam shall be applied to the deep layers of Ciliary Iris area at 532 nanometer wavelength, 2000 micrometer spot diameter in the same session,
i—When the number of pigments shedding from the superficial layer of ciliary iris to the front chamber is more than 25 large particles or 100 small cells, don't apply 2000 micrometer spot diameter laser beam at 532 nanometer wavelength to the deep layers of Ciliary Iris area in the same session, pause the laser operation and irrigate the iris pigments in the front chamber through Irrigation Aspiration (I/A) under sterile conditions with BSS Plus or isolyte solution
j—Application of respectively 1000 and 2000 micrometers spot diameter laser beam with 532 nanometer wavelength in Pupillary and Ciliary area after I/A, consists of the steps above and takes approximately five to ten minutes per eye.

2. The method mentioned in 1st claim the iris color changing method, the characteristics: after the process steps consisting of a, b, c, d, e, f, g, h, i and j, a session; after a, b, c, d, e, f, g, h, i and j process steps, if there are pigmented areas in the superficial layer and/or stroma of iris in biomicroscopic examination on the eye applied with steps a, b, c, d, e, f, g, h, i and j, the session is repeated until the pigmented areas disappear.
